(19) United States
(12) Patent Application Publication
(10) Pub. No.: US 2014/0356449 A1
(43) Pub. Date: Dec. 4, 2014

(54) SORE THROAT REMEDY

(71) Applicant: Edward Babinski, Saint Johns, MI (US)

(72) Inventor: Edward Babinski, Saint Johns, MI (US)

(21) Appl. No.: 14/291,054

(22) Filed: May 30, 2014

Related U.S. Application Data
(60) Provisional application No. 61/830,714, filed on Jun. 4, 2013.

(51) Int. Cl. A61K 35/32; A23L 2/02; A61K 9/00

(52) U.S. Cl. CPC A61K 35/32; A61K 9/0056; A23L 2/02; A23V 2002/00
USPC 424/548

(57) ABSTRACT

A method for treating a sore throat involves administering to a human subject in need of treatment of at least one of cartilage and a material derived from cartilage in a vehicle or composition that promotes or facilitates prolonged or extended contact of a sore throat with at least one of cartilage and a material derived from cartilage to reduce or eliminate the symptoms of a sore throat, typically within hours. The cartilage or materials derived from cartilage can be administered in a powdered form that is dispersed in a beverage, a syrup or a lozenge.

### CROSS-REFERENCE TO RELATED APPLICATIONS

This application claims priority to U.S. provisional application Ser. No. 61/830,714, entitled “Sore Throat Remedy,” filed Jun. 3, 2013.

### FIELD OF THE DISCLOSURE

This disclosure relates to methods, kits, and dosage forms for treating a sore throat by contacting an irritated throat with a material that has the effect of alleviating discomfort and symptoms associated with a sore throat.

### BACKGROUND OF THE DISCLOSURE

A sore throat is characterized by a pain or irritation of the throat or pharynx, usually caused by acute pharyngitis. A sore throat is most often caused by a viral infection. A sore throat can also be caused by a streptococcal infection, tumors, gastroesophageal reflux disease, mononucleosis, and allergies.

A sore throat can develop for a number of reasons including a viral or bacterial infection, or a common or seasonal allergy. Often associated with an infection, common or seasonal allergy is some degree of nasal or sinus congestion. This congestion is typically referred to as post-nasal drip, in which mucous originating on the surface of the nasal mucosa or the sinus mucosa drains onto the upper esophagus. The accumulation of nasal mucosa in the upper esophagus also stimulates the swallowing reflex often associated with a sore throat. The swallowing reflex transports the acidic mucous into relatively constant contact with the region of the throat. The acidic nature of the mucous from the sinus mucosa or nasal mucosa erodes the epithelial tissue of the throat thereby exposing the underlying tissue to the acidic mucous. The nerve endings in the underlying tissue in contact with the acidic mucosa cause what we identify as the discomfort or pain associated with a sore throat. The more inflamed the nasal mucosa or the sinus mucosa, the greater the production of the acidic mucous, the greater the erosion and the greater the severity of the pain and discomfort associated with the sore throat.

Conventional treatments or remedies for a sore throat include gargling with warm salt water to reduce swelling and relieve discomfort; administration of throat lozenges or cough drops; administration of drugs such as aspirin, acetaminophen, ibuprofen or naproxen, to reduce fever or pain; and administration of a decongestant to reduce swelling of mucous membranes and to relieve post-nasal drip.

Sore throat lozenges and cough drops have comprised anesthetic compounds, such as menthol benzocaine; antiseptic compounds, such as eucalyptus oil; oral demulcent compounds, such as pectin; and antitussives, such as dextromethophan. Certain throat lozenges are also known to contain ingredients such as peppermint oil, spearmint or honey, which may provide a soothing effect.

Bovine and shark cartilage supplements have been proposed as treatments for cancer. Chondroitin derived from bovine or shark cartilage has been publicized as a treatment for osteoarthritis, and has been administered in pill, powder and liquid oral dosage forms. Cartilage and materials derived from cartilage (e.g., shark or bovine) have also been proposed or used in attempts to treat psoriasis using oral dosage forms, wound healing using topical compositions, and for maintaining or improving eye health using oral dosage forms.

### SUMMARY OF THE DISCLOSURE

The disclosed treatments for a sore throat involve oral administration of at least one of cartilage and a material derived from cartilage in a vehicle or composition that promotes or facilitates prolonged or extended contact of a sore throat with at least one of cartilage and a material derived from cartilage to reduce or eliminate the symptoms of a sore throat, typically within hours.

The methods disclosed for treating a sore throat include a step of administering to a human in need of treatment for a sore throat an oral dosage form that facilitates contact between an irritated throat and a liquid containing at least one of cartilage and a material derived from cartilage.

### DESCRIPTION OF THE DRAWINGS

FIG. 1 is a flowchart illustrating a method of providing sore throat relief in accordance with one or more of the less viscous liquid embodiments of the invention.

FIG. 2 is a flowchart illustrating a method of providing sore throat relief in accordance with one or more of the more viscous liquid embodiments of the invention.

FIG. 3 is a flowchart illustrating a method of providing sore throat relief in accordance with one or more of the lozenge embodiments of the invention.

### DETAILED DESCRIPTION

One or more of the embodiments of the present invention relate to the oral administration of at least one of cartilage and a material derived from cartilage such as collagen fibers, partially hydrolyzed collagen fibers, elastin fibers, and proteoglycans, in a vehicle or composition that promotes or facilitates prolonged or extended contact of a sore throat with at least one of cartilage and a material derived from cartilage to reduce or eliminate the symptoms of a sore throat. The cartilage or material derived from cartilage can be from any source but preferably from sharks or bovine animals. The cartilage or material derived from cartilage can be in a solid form, such as a powder or granulated form, or in a liquid form (e.g., liquid collagen). The cartilage or material derived from cartilage can be added to a dosage form such as dispersing the material in a liquid, including the material in a kit, or incorporating the material in to a cough drop or lozenge. Any method of oral administration of at least one of cartilage and a material derived from cartilage that creates extended contact with the throat may be used for the same purpose and should be considered embodiments of the invention.

Examples of liquids in which the cartilage and materials derived from cartilage may be dispersed include water, fruit juices, teas, other beverages, and syrups. When the treatment involves administering a dosage form comprising cartilage or cartilage-derived materials dispersed in a less viscous liquid, slow sipping of the beverage facilitates prolonged contact of the cartilage or material derived from cartilage with the throat.

When the treatment involves administration of a lozenge or cough drop dosage form the cartilage or material derived from cartilage is dispersed in a continuous matrix comprising an edible carrier material. The edible carrier material may be selected from at least one of sugars, polyethylene glycol, chocolate, gelatin and gums. As is well known, such dosage forms are typically formulated from sugar, honey or other materials that can induce salivation allowing a slow erosion or dissolution of the lozenge or cough drop to cause the cartilage or material derived from cartilage to be released into the saliva and facilitate prolonged contact of the cartilage or material derived from cartilage with the throat. When the treatment involves administration of a lozenge or cough drop dosage form the liquid in which the cartilage or material derived from cartilage is dispersed is a human subject's saliva.

When the treatment involves administration of a kit dosage form the kit may comprise at least one of cartilage and a material derived from cartilage in a dry powder form, along with instructions to disperse the powder in a liquid beverage and drink the resulting dispersion. Instructions may require the administration of multiple doses in spaced apart time intervals of about to 5 hours. Kits may also be comprised so that the dry powder is contained in one or a plurality of sealed packets prior to use. Each packet may contain a single dose of dry power. Preferably, dosages may range from 100 to 2000 milligrams.

The dry powder in the kit can be contained in a sealed packet that can be torn open prior to use, and which contains a single dose that can be dispensed in a liquid beverage for a single treatment session. The dry powder may include components other than cartilage or material derived from cartilage such as a flavoring that may be dispensed in water.

A single dose of the cartilage or material derived from cartilage can be 5 milligrams to 1000 milligrams. However, doses greater than 1000 milligrams would not be expected to be harmful, and could actually be beneficial. It is also expected that some benefit can result from doses less than 5 milligrams.

It is possible that beneficial results can be achieved with a single dose administered in a single treatment session. However, multiple treatment sessions (e.g., 2 or 3) spaced apart by a time interval from about ½ hour to 5 hours can also provide beneficial results.

When lozenge forms are employed, the lozenge can be a soft, hard, or chewable lozenge in which the cartilage or material derived from cartilage is dispersed, such as in a finely divided form (e.g., powder) in an edible, solid or semi-solid matrix comprised of materials such as sugars, polyethylene glycol, chocolate, gelatin or gums. As used herein, the term “sore throat” refers to a condition in which a human experiences inflammation, swelling, pain or other discomfort of the throat, regardless of cause. The term “treating” refers to performing a method or procedure that includes administering cartilage or material derived from cartilage to a human experiencing a sore throat to reduce or eliminate symptoms and discomfort associated with the sore throat, and therefore encompasses complete or partial alleviation of the symptoms and discomfort. Preferably, lozenge dosage forms contains from 1 milligram to 1000 milligrams of the at least one of cartilage and a material derived from cartilage.

Over a ten year period, three subjects were treated for sore throats with 500 milligrams of cartilage added to a liquid (heated apple juice). The subjects would slowly sip the mixture over a 5-15 minute time period, stirring the mixture before each sip to remix the cartilage with the liquid. Relief for a minor sore throat was usually achieved after one dose within two hours. If satisfactory results were not achieved with a single dose, then a second dose would be given two hours later. Severe sore throats may take 3 or 4 doses to achieve desired affects. During an active cold, or other viral infection including a sore throat, it may be necessary to take additional doses on an as needed basis. Usually one or two doses in a 24-hour period is sufficient.

Subjects averaged two sore throats a year for a total of 60. Only the first sore throat treated was severe, and required four doses to achieve desired results, with increased reduction in pain after each dose. All other cases were mild to moderate.

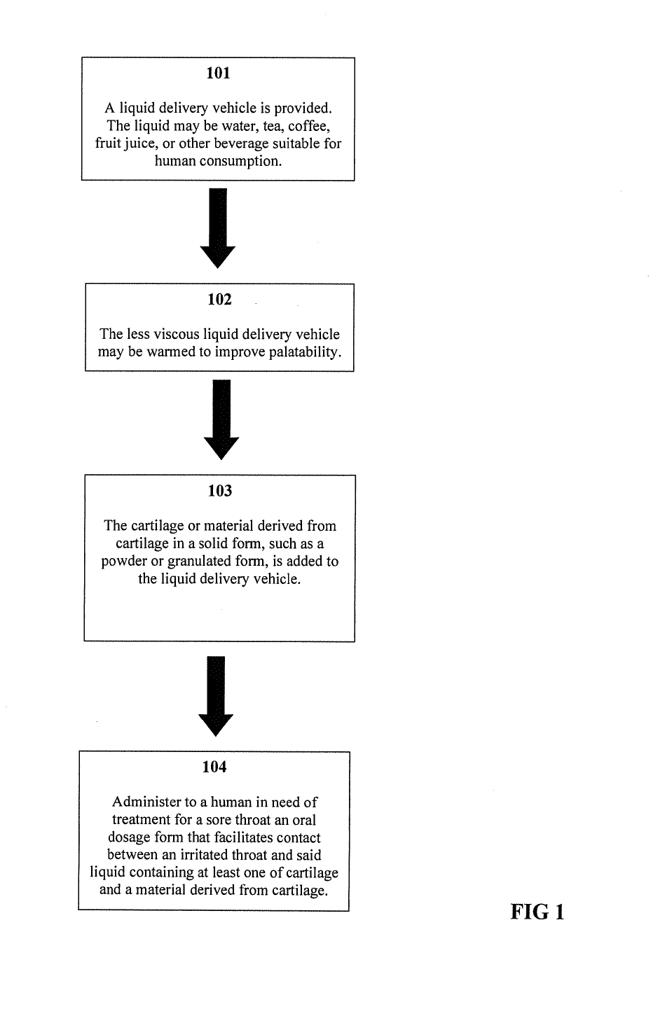

As illustrated in the flow chart of FIG. 1, a method of providing sore throat relief in accordance with one or more of the less viscous liquid embodiments of the invention disclosed herein involves the following steps. In step 101 a liquid delivery vehicle is provided. The liquid delivery vehicle may be water, tea, coffee, or fruit juice, or other beverage suitable for human consumption. Examples of suitable fruit juices that may be used as the beverage in which the cartilage is dispersed include apple juice, orange juice, grape juice, cranberry juice, lemon juice, lime juice, cherry juice, grapefruit juice, pineapple juice, etc. While not being bound by a particular theory, it is believed that the tannin components in some beverages, act as a mucoadhesive to hold the cartilage in the throat to promote healing of the effected tissue.

In step 102, the less viscous liquid delivery vehicle may be warmed to improve palatability. Such beverages can be heated for administration at a temperature of at least 100° F., 120° F., 140° F., 160° F. or 180° F. The temperature of the liquid delivery vehicle may be varied depending on the nature of the liquid delivery vehicle. For example, fruit juices may be warmed to a lesser extent than tea in order to prevent scalding of the beverage.

In step 103, cartilage or material derived from cartilage in a solid form, such as a powder or granulated form, is added to the liquid delivery vehicle. The cartilage and the materials derived from cartilage are not typically completely soluble in water. Thus, the term “dispersed” in a liquid vehicle or carrier is meant to encompass suspended, even if temporary. In order to promote suspension of insoluble materials, it is desirable to utilize cartilage and materials derived from cartilage that are ground into a fine powder that settles slowly. The term “dispersed” has also been used herein to refer to cartilage and materials derived from cartilage that are contained in solid lozenge form. In such case, “dispersed” means that the cartilage or materials derived from cartilage are in a divided (e.g., powdered) form and are relatively uniformly distributed in a continuous solid phase that constitutes the carrier or matrix from which the cartilage or material derived from cartilage is released during use of the lozenge. Although not required, in preferred methods the cartilage is stirred into the less viscous liquid delivery vehicle to promote greater dispersion.

Examples of materials derived from cartilage include collagen fibers; elastin fibers; proteoglycans; partially hydrolyzed collagen fibers; proteins such as troponin-I, tetranectin-type protein, collagenases, cartilage-derived inhibitor (CDI), tissue inhibitors of metalloproteinases (TIMPs); glycoproteins such as shyrnastatin-1 and -2, galactosamine, glucosamine; glycosaminoglycans such as chondroitin sulfate-D, chondroitin-6-sulfate and keratin sulfate; which may be employed individually or in any combination, with or without cartilage.

There is considerable flexibility with the amount of cartilage or material derived from cartilage that can be employed in a single dose used in a single therapy session. While even small amounts of cartilage or derivatives of cartilage brought into contact with the throat are expected to provide a beneficial result, an effective therapeutic dose for a single treatment session is at least about 20 milligrams, 50 milligrams or 100 milligrams.

In step 104, the resulting mixture is administered to a human in need of treatment for a sore throat by oral ingestion. Ingesting the mixture in a way that promotes or facilitates contact of the cartilage or material derived from cartilage with the affected throat tissue is recommended but not required. As such, ingestion in a manner that allows the mixture to reside in the oral cavity for a prolonged period of time, e.g., 30 seconds to 15 minutes, 1 minute to 10 minutes, or 2 minutes to 5 minutes is preferred. When the vehicle or carrier is a less viscous liquid, such as water, fruit juices or other common beverages, prolonged or extended contact with the cartilage or material derived from cartilage can be better facilitated by instructing the human subject in need of treatment to gargle or slowly sip the liquid dosage form. Simply, ingesting cartilage in tablet forms that are intended to be swallowed before release of the cartilage or material derived from cartilage would not be expected to have the desired therapeutic effect.

A particularly efficacious composition for treating sore throats is a combination of shark cartilage and heated apple juice. While not being bound to a particular theory or mechanism, it is believed that when shark cartilage is mixed with apple juice some of the tannins in the juice bind to the cartilage. When consumed, the tannins coating the powdered cartilage bind the salivary proteins in the throat forming a protective barrier. Testing has shown that tannin levels in apple juice drop as the cartilage settles out of the mixture. Liquids containing high levels of tannins should be diluted to minimize the astringency from the tannins that cause a dry feeling in the mouth. The astringency of the mixture increases with time (e.g. 0-60 minutes) and may be significantly stronger than consuming the liquid by itself. The proper mixture should cause very little drying of the mouth and throat.

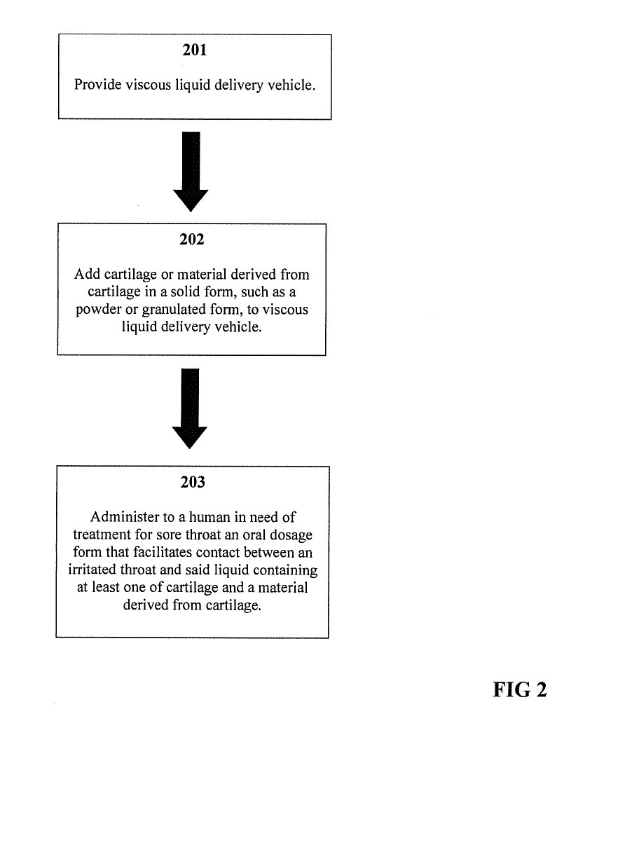

As illustrated in the flow chart of FIG. 2, a method of providing sore throat relief in accordance with one or more of the more viscous liquid embodiments of the invention disclosed herein involves the following steps. In step 201 a viscous liquid delivery vehicle is provided. Syrups and other highly viscous liquids are useful vehicles or carriers for delivering the cartilage or material derived from cartilage to the throat because of their tendency to develop a sustained coating on the tissue which facilitates contact with the cartilage or material derived from cartilage.

In step 202, cartilage or material derived from cartilage in a solid form, such as a powder or granulated form, is added to the viscous liquid delivery vehicle. Although not required, in preferred methods the cartilage is stirred into the liquid delivery vehicle to promote greater dispersion.

In step 203, the resulting mixture is administered to a human in need of treatment for a sore throat by oral ingestion. The methods, kits and dosage forms disclosed herein may also be supplemented with vitamins (e.g., vitamin C) and minerals to improve overall health and resistance to infections that can induce a sore throat.

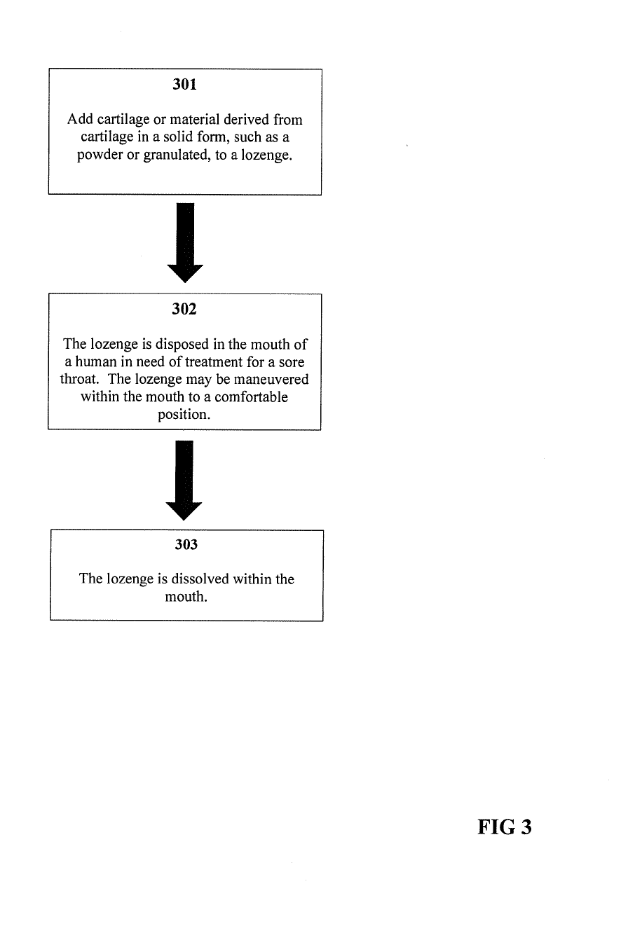

As illustrated in the flow chart of FIG. 3, a method of providing sore throat relief in accordance with one or more of the lozenge embodiments of the invention disclosed herein involves the following steps. The term “lozenge” is intended to encompass any throat lozenge, cough drop or other solid dosage form that is intended to reside in the oral cavity for a prolonged or extended period of time to slowly release cartilage or a material derived from cartilage, and includes soft, hard and chewable lozenges or drops. In step 301 a lozenge containing cartilage or material derived from cartilage in a solid form is provided.

In step 302, the lozenge is disposed in the mouth of a human in need of treatment for a sore throat. The lozenge may be maneuvered within the mouth to a comfortable position.

In step 303, the lozenge is dissolved within the mouth. In one or more embodiments, the lozenge may be designed to dissolve slowly. Dissolution time is a function of the lozenge size. When dissolved, the components of the lozenge may comprise a runny consistency, which may reach the throat.

The therapeutic methods, dosage forms and kits disclosed herein may include an oral spray or the use of an oral spray, which may be administered via a spray nozzle or a pump spray bottle or a spray bottle containing an inert propellant suitable for human consumption.

While beneficial results can be achieved with a single treatment session, excellent results are expected with two, three or four sessions approximately 1 hour to 4 hours apart, e.g., 1 to 3 hours, or 2 to 3 hours apart.

The dosage forms and therapeutic methods described herein may be useful for treatment of a sore throat caused by allergens, chemicals, acids, alkaloids, hot liquids, gases, erosion of the esophagus due to acid reflux, ulcers of the throat, bacterial and viral infections of the throat, cold and flu viruses, and strep throat (streptococcal pharyngitis). The dosage forms and therapeutic methods may offer some protection from contracting strep throat, provide treatment for bacterial and viral infections of the sinuses, and offer some protection from contracting an infection. Other possible uses include improving oral health, and the treatment of stomach ailments such as indigestion, nausea, heartburn, ulcers, bacterial and viral infections, protecting the surfaces of the mouth and the stomach lining in the same manner as the throat.

While the foregoing describes various embodiments of the invention, other and further embodiments of the invention may be devised without departing from the basic scope thereof. The scope of the invention is determined by the claims that follow. The invention is not limited to the described embodiments, versions, or examples, which are included to enable a person having ordinary skill in the art to make and use the invention when combined with information and knowledge available to the person having ordinary skill in the art.

What is claimed:

1. A method for treating a sore throat, comprising administering to a human in need of treatment for a sore throat a dosage form that facilitates contact between an irritated throat and a liquid containing at least one of cartilage and a material derived from cartilage.

2. The method of claim 1, in which the cartilage or at least one material derived from cartilage comprises shark cartilage or at least one material derived from shark cartilage.

3. The method of claim 1, in which the cartilage or materials derived from cartilage comprises shark cartilage.

4. The method of claim 3, in which the shark cartilage is in a powder form that is dispersed in the dosage form.

5. The method of claim 1, in which the cartilage or material derived from cartilage comprises at least one of the materials selected from the group consisting of collagen fibers, partially hydrolyzed collagen fibers, elastin fibers, and proteoglycans.

6. The method of claim 1, in which the dosage form is a more viscous liquid in which the cartilage or material derived from cartilage is dispersed.

7. The method of claim 1, in which the dosage form is a lozenge, and the liquid is saliva.

8. The method of claim 1, in which the dosage form is a less viscous liquid.

9. The method of claim 8, wherein administering to a human in need of treatment for a sore throat a dosage form that facilitates contact between an irritated throat and a liquid containing at least one of cartilage and a material derived from cartilage includes:
providing a liquid delivery vehicle;
adding cartilage or material derived from cartilage in a solid form, such as a powder or granulated form, to a liquid; and
instructing a human in need of treatment for sore throat to ingest the liquid containing at least one of cartilage and a material derived from cartilage in a manner that facilitates contact between an irritated throat.

10. The method according to claim 9, wherein administering to a human in need of treatment for a sore throat a dosage form that facilitates contact between an irritated throat and a liquid containing at least one of cartilage and a material derived from cartilage further includes:
warming the liquid delivery vehicle containing cartilage or material derived from cartilage.

11. The method of claim 10, in which the dosage form is a fruit juice in which the cartilage or material derived from cartilage is dispersed.

12. The method of claim 10, in which the dosage form is prepared by dispersing the at least one of cartilage and a material derived from cartilage in a beverage liquid that has been heated and administered at a temperature above 100° F.

13. The method of claim 1 in which the dosage form comprises a liquid containing at least one of cartilage and a material derived from cartilage, and at least one of apple juice and a material derived from apple juice.

14. A kit for treating a sore throat, comprising at least one of cartilage and a material derived from cartilage in a dry powder form, and instructions to disperse the dry powder in a liquid beverage and drink the resulting dispersion.

15. The kit of claim 14, in which the dry powder is contained in one or a plurality of sealed packets prior to use.

16. The kit of claim 15, in which each packet contains a single dose of dry powder.

17. The kit of claim 16, in which each single dose comprises 100 milligrams to 2000 milligrams of the dry powder.

18. The kit of claim 16, in which the instructions require administration of multiple doses in spaced apart time intervals of about ½ hour to 5 hours.

19. A dosage form for treating a sore throat, comprising a lozenge having at least one of cartilage and a material derived from cartilage dispersed in a continuous matrix comprising an edible carrier material.

20. The dosage form of claim 19, in which the edible carrier material is selected from at least one of sugars, polyethylene glycol, chocolate, gelatin and gums.

21. The dosage form of claim 19, in which the lozenge contains from 1 milligram to 1000 milligrams of the at least one of cartilage and a material derived from cartilage.

22. The dosage form of claim 19, in which the edible carrier material is selected from at least one of sugars, polyethylene glycol, chocolate, gelatin and gums; wherein the lozenge contains from 1 milligram to 1000 milligrams of the at least one of cartilage and a material derived from cartilage; and wherein the at least one of cartilage and a material derived from cartilage comprises shark cartilage or at least one material derived from shark cartilage.

23. The method of claim 7, wherein administering to a human in need of treatment for a sore throat a dosage form that facilitates contact between an irritated throat and a liquid containing at least one of cartilage and a material derived from cartilage includes:
adding cartilage or material derived from cartilage in a solid form, such as a powder or granulated, to a lozenge;
disposing the lozenge in the mouth of a human in need of treatment for sore throat;
maneuvering the lozenge within the mouth to comfortable position; and
dissolving the lozenge within the mouth.

24. The method of claim 6, wherein administering to a human in need of treatment for a sore throat a dosage form that facilitates contact between an irritated throat and a liquid containing at least one of cartilage and a material derived from cartilage includes:
providing a viscous liquid delivery vehicle;
adding cartilage or material derived from cartilage in a solid form;
stirring the viscous liquid delivery vehicle to promote greater dispersion of the cartilage or material derived from cartilage in a solid form.
instructing a human in need of treatment for sore throat to ingest the liquid containing at least one of cartilage and a material derived from cartilage in a manner that facilitates contact between an irritated throat.
